(19) United States
(12) Patent Application Publication
(10) Pub. No.: US 2026/0183454 A1
(43) Pub. Date: Jul. 2, 2026

(54) DEVICES FOR REDUCING IMPLANT ASSOCIATED INFECTIONS

(71) Applicant: ZnAble LLC, Northport, MI (US)

(72) Inventors: Jayne Robinson, Northport, MI (US); James E. Kemler, Glen Arbor, MI (US)

(73) Assignee: ZnAble LLC, Northport, MI (US)

(21) Appl. No.: 19/549,637

(22) Filed: Feb. 25, 2026

Related U.S. Application Data
(63) Continuation of application No. PCT/US25/45882, filed on Sep. 11, 2025.
(60) Provisional application No. 63/693,848, filed on Sep. 12, 2024.

(51) Int. Cl. A61L 27/54; A61L 27/04; A61L 27/06; A61L 27/10; A61L 31/02; A61L 31/16

(52) U.S. Cl. CPC A61L 27/54; A61L 27/045; A61L 27/047; A61L 27/06; A61L 27/10; A61L 31/022; A61L 31/026; A61L 31/16; A61L 2300/406; A61L 2300/408

(57) ABSTRACT

A method of reducing biofilm formation on an implant optionally includes soaking an orthopedic or dental implant comprising a biocompatible material in an infection control composition comprising a porphyrin. An implant for implantation in an animal body optionally includes an implant body comprising a biocompatible material and defining one or more reservoirs; and an infection control composition comprising a porphyrin composition configured to be retained in the one or more reservoirs, wherein the one or more reservoirs are configured to elute the porphyrin composition over time.

### CROSS-REFERENCE TO RELATED APPLICATIONS

This application is a continuation of International Patent Application Ser. No. PCT/US2025/045882, filed Sep. 11, 2025; which claims the priority benefit of U.S. Provisional Patent Application Ser. No. 63/693,848, filed Sep. 12, 2024, the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of infection control or prevention and, more specifically, to the field of implant infection control or prevention. Described herein are compositions, devices, and methods for reducing implant associated infections.

### BACKGROUND

Implant-associated infections, particularly those related to orthopedic and dental implants, are a significant concern in medical practice. These infections occur when bacteria or other pathogens colonize the surface of the implant or the surrounding tissues, leading to inflammation, pain, and potentially severe complications if left untreated. There are several causes and risk factors for implant-associated infections. For example, during the implantation surgery, despite rigorous sterile procedures, bacteria from the skin or surrounding environment can contaminate the implant. Bacteria can also reach the implant through the bloodstream from other infected sites in the body. Once bacteria adhere to the surface of the implant, they can form biofilms or resilient colonies that are resistant to antibiotics and immune responses. Factors such as diabetes, immunosuppression, obesity, and smoking can increase the risk of infection by impairing the body's ability to fight off bacteria.

There are several types of infections. Early infections typically occur within the first few weeks to months after surgery. Delayed infections can occur months to years after surgery, often due to hematogenous spread or latent biofilm formation. Further, acute infections can present with a rapid onset of symptoms, while chronic infections may have subtle symptoms and are often associated with biofilm formation. Symptoms of implant-associated infections can include persistent pain around the implant site; swelling, warmth, or redness; fever and chills; wound drainage or pus; and/or limited range of motion.

Current treatment modalities include antibiotics (e.g., administered orally or intravenously), surgical intervention (e.g., debride infected tissues, remove the implant, and/or implant a new device), and/or biofilm disruption (e.g., antibiotics combined with agents that break down the biofilm matrix). Current preventative measures include preoperative preparation (e.g., proper sterilization techniques and minimizing contamination during surgery); antibiotic prophylaxis (e.g., administering antibiotics before surgery to reduce the risk of infection); and/or postoperative monitoring.

### SUMMARY

In some embodiments, the techniques described herein relate to a method of reducing biofilm formation on an implant, including: soaking an orthopedic or dental implant including a biocompatible material in an infection control composition including a porphyrin.

In some embodiments, the techniques described herein relate to a method, wherein the orthopedic implant is formed by one of: sintering, casting, injection molding, or machining.

In some embodiments, the techniques described herein relate to a method, wherein the biocompatible material includes one or more of: a stainless steel, a titanium alloy, a ceramic, a cobalt chromium alloy, an oxidized zirconium alloy, bone cement, polyethylene, or a polymer.

In some embodiments, the techniques described herein relate to a method, wherein the orthopedic implant includes one of: a joint implant, a bone plate, an intramedullary rod, a bone graft, a spinal implant, or a dental implant.

In some embodiments, the techniques described herein relate to a method, wherein the porphyrin includes:

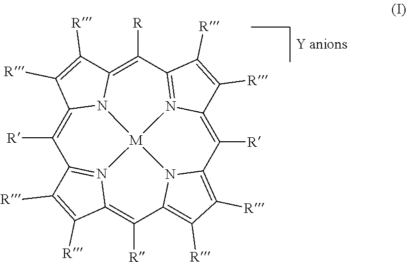

wherein, M is a transition metal (II) cation, R is a mono-, di-, tri-, tetra-, or penta-halophenyl and the halogen is selected from the group consisting of Cl, F, Br, and combinations thereof, R′ is

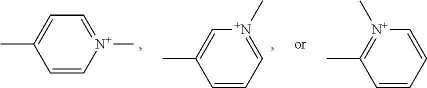

R″ is equal to R or R′, R′″ is —H, —F, —Cl, or —Br, and Y is 2 or 3.

In some embodiments, the techniques described herein relate to a method, wherein the transition metal M is a Fe⁺², a Co⁺², a Ni⁺², or Zn⁺².

In some embodiments, the techniques described herein relate to a method, wherein the porphyrin includes:

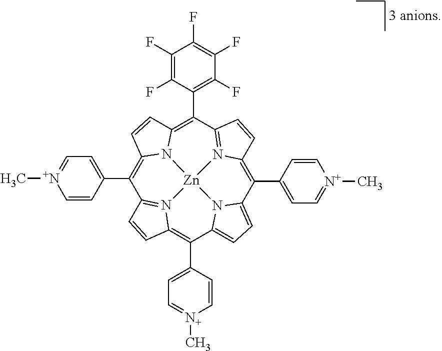

and wherein the anion is p-toluene sulfonate.

In some embodiments, the techniques described herein relate to a method, wherein the porphyrin includes a transition metal porphyrin complex having, in an absence of light, one or more of: antimicrobial activity, antibacterial activity, or antiviral activity.

In some embodiments, the techniques described herein relate to a method, wherein the infection control composition further includes a pharmaceutically acceptable excipient.

In some embodiments, the techniques described herein relate to a method, wherein a weight ratio of the porphyrin to the pharmaceutically acceptable excipient is about 1:1,000 to about 1:500,000.

In some embodiments, the techniques described herein relate to a method, wherein a weight ratio of the porphyrin to the pharmaceutically acceptable excipient is about 1:2,000 to about 1:250,000.

In some embodiments, the techniques described herein relate to a method, wherein the pharmaceutically acceptable excipient includes one or more of: saline, water, a disinfectant, or a combination thereof.

In some embodiments, the techniques described herein relate to a method, wherein an effective concentration of the porphyrin in the infection control composition is about 2 μg/mL to about 1000 μg/mL.

In some embodiments, the techniques described herein relate to a method, wherein an effective concentration of the porphyrin in the infection control composition is about 4 μg/mL to about 500 μg/mL.

In some embodiments, the techniques described herein relate to a method, wherein the orthopedic implant is light activated before implantation.

In some embodiments, the techniques described herein relate to a method, wherein the light has a wavelength of about 400 nm to about 850 nm.

In some embodiments, the techniques described herein relate to a method, further including reducing biofilm formation on the implant.

In some embodiments, the techniques described herein relate to an implant for implantation in an animal body, including: an implant body including a biocompatible material and defining one or more reservoirs; and an infection control composition including a porphyrin composition configured to be retained in the one or more reservoirs, wherein the one or more reservoirs are configured to elute the porphyrin composition over time.

In some embodiments, the techniques described herein relate to an implant, wherein the biocompatible material includes one or more of: a stainless steel, a titanium alloy, a ceramic, a cobalt chromium alloy, an oxidized zirconium alloy, bone cement, polyethylene, or a polymer.

In some embodiments, the techniques described herein relate to an implant, wherein the implant includes one of: a joint implant, a bone plate, an intramedullary rod, an intramedullary nail, a bone graft, a spinal implant, or a dental implant.

In some embodiments, the techniques described herein relate to an implant, wherein the porphyrin includes:

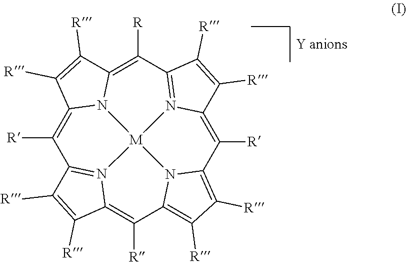

wherein, M is a transition metal (II) cation, R is a mono-, di-, tri-, tetra-, or penta-halophenyl and the halogen is selected from the group consisting of Cl, F, Br, and combinations thereof, R′ is

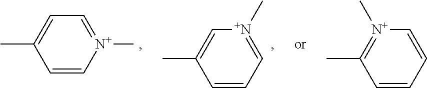

R″ is equal to R or R′, R′″ is —H, —F, —Cl, or —Br, and Y is 2 or 3.

In some embodiments, the techniques described herein relate to an implant, wherein the transition metal Mis a Fe⁺², a Co⁺², a Ni⁺², or Zn⁺².

In some embodiments, the techniques described herein relate to an implant, wherein the porphyrin composition includes:

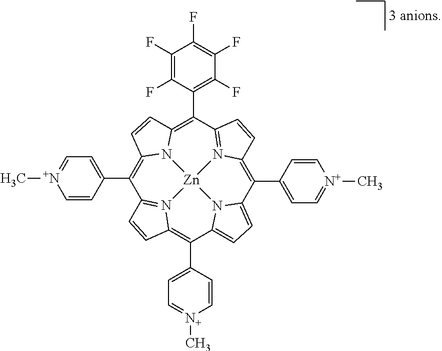

wherein the anion is p-toluene sulfonate.

In some embodiments, the techniques described herein relate to an implant, wherein the porphyrin includes a transition metal porphyrin complex having, in an absence of light, one or more of: antimicrobial activity, antibacterial activity, or antiviral activity.

In some embodiments, the techniques described herein relate to an implant, wherein a volume of a reservoir of the one or more reservoirs defined by the implant body is about 1 mL to about 10 mL.

In some embodiments, the techniques described herein relate to an implant, wherein a concentration of the porphyrin composition in the infection control composition is about 2 μg/mL to about 1000 μg/mL.

In some embodiments, the techniques described herein relate to an implant, wherein a concentration of the porphyrin composition in the infection control composition is about 4 μg/mL to about 500 μg/mL.

In some embodiments, the techniques described herein relate to an implant, wherein an external surface of the implant body is substantially resistant to biofilm formation for about one week to about six months.

In some embodiments, the techniques described herein relate to an implant, wherein the implant body is porous.

In some embodiments, the techniques described herein relate to an implant, wherein a porosity of the implant body is about 30% to about 80%.

In some embodiments, the techniques described herein relate to an implant for implantation in an animal body, including: an implant body including a biocompatible material, the implant body having a hydroxyapatite coating on at least a portion of an exterior surface of the implant body; and an infection control composition including porphyrin configured to react with or bond to the hydroxyapatite coating, wherein the porphyrin is configured to reduce biofilm formation on the implant body.

In some embodiments, the techniques described herein relate to a method of reducing biofilm formation on an implant, including: reaming an intramedullary canal of a bone; applying an infection control composition including porphyrin to the reamed intramedullary canal; implanting an intramedullary nail into the intramedullary canal; and reducing infection or biofilm formation on the intramedullary nail.

In some embodiments, the techniques described herein relate to a method of manufacturing an implant, including: mixing a biocompatible material with an infection control composition including porphyrin to form a mixture; and casting the mixture in a mold to form an orthopedic implant.

In some embodiments, the techniques described herein relate to a method of manufacturing an implant including: mixing a biocompatible material with an infection control composition including porphyrin to form a mixture; and additively manufacturing an implant using, at least in part, the mixture.

In some embodiments, the techniques described herein relate to a method, wherein the additively manufacturing is selective laser melting, electron beam melting, stereolithography, or fused deposition modeling.

In some embodiments, the techniques described herein relate to a method of disinfecting an implant, including: applying an infection control composition to an external surface of an orthopedic implant, the infection control composition including a porphyrin and a pharmaceutically acceptable excipient.

In some embodiments, the techniques described herein relate to a method, further including activating the porphyrin with light having a wavelength between about 400 nm to about 850 nm.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 shows a schematic of an x-ray image of an embodiment of a bone screw anchored in a bone.

FIG. 2 shows a schematic of an x-ray image of an embodiment of a bone plate anchored to a bone.

FIG. 3A shows an embodiment of an orthopedic implant.

FIG. 3B shows an embodiment of an orthopedic implant, the acetabular portion of the implant defining a reservoir.

FIG. 3C shows an embodiment of an orthopedic implant being sprayed with an infection control composition.

FIG. 3D shows an embodiment of an orthopedic implant, the femoral head portion of the implant defining a reservoir.

FIG. 3E shows an embodiment of an orthopedic implant, the stem portion of the implant defining a reservoir.

FIG. 3F shows an embodiment of an orthopedic implant being sprayed with a light-activated infection control composition.

FIG. 4 shows a side view of an embodiment of an intramedullary nail.

FIG. 5A shows a perspective view of a dental implant.

FIG. 5B shows a perspective view of a dental implant defining a reservoir.

FIGS. 6A-6D show an embodiment of a method of applying bone graft material, including an infection control composition, to a bone.

FIG. 7 shows a receptacle sized and shaped to receive a biomedical device therein for applying an infection control composition to the biomedical device.

FIG. 8 shows an embodiment of a biomedical device having an infection control coating thereon.

FIGS. 9A-9C shows an embodiment of a method for applying an infection control composition to a bone reaming process and/or bone reamings.

FIGS. 10A-10D show a proposed model of the mechanism of action of ZnPor against Pseudomonas aeruginosa (PsA) biofilms and individual cells.

FIGS. 11A-11C show experimental data of the distribution and the concentration-dependent effect of ZnPor against PsA PAO1 biofilm.

FIG. 12 shows experimental data of PsA PAO1 biofilms treated with ZnPor and PEV2 alone and in combination without photoactivation.

FIG. 13 shows experimental data in table format of PAO1 Biofilms treated with ZnPor in the presence and absence of photoactivation.

FIG. 14 shows prophetic elution profile data of ZnPor concentration over time.

FIG. 15 shows an embodiment of a method of casting an infection resistant implant.

FIG. 16 shows an embodiment of a method of additively manufacturing an infection resistant implant.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Orthopedic implants offer several benefits, particularly in the context of treating musculoskeletal injuries and conditions. Implants can restore lost function to joints or bones that have been damaged by injury or disease. For example, hip or knee implants can significantly improve mobility and quality of life for patients with injuries or severe arthritis. Further, by stabilizing joints or bones, implants can alleviate pain that was previously caused by movement or instability. This is particularly important in conditions like fractures or degenerative joint diseases. Many orthopedic implants allow patients to return to their normal activities and lifestyles, which can greatly enhance their overall quality of life. This includes activities such as walking, exercising, or performing daily tasks without discomfort. Conventional orthopedic implants are designed to be durable and long-lasting, providing reliable support over many years. This durability reduces the need for frequent replacements or revisions. Further, implants can be customized to fit each patient's unique anatomy and needs. This personalization ensures a better fit and function, leading to improved outcomes and reduced complications. In cases of severe fractures or bone loss, implants can provide stability and support that facilitate the natural healing process. This may include promoting bone growth or fusion around the implant. Additionally, advances in technology have led to minimally invasive surgical techniques for implant placement. These procedures often result in smaller incisions, less tissue damage, reduced pain, and faster recovery times for patients. Orthopedic implants have been extensively studied and refined, leading to predictable outcomes for many patients. Surgeons can often anticipate how well an implant will function based on its design and the patient's condition.

Despite all the benefits of orthopedic implants, infections associated with the implants can be life-threatening. In developed countries, the rate of infection can range from about 2% to 10%, while in developing regions it can be as high as 15%. In the U.S., there are more than 100,000 cases of orthopedic implant-associated infections (ODRIs) each year, which can cost the healthcare system billions of dollars. About 6% of ODRIs can lead to intensive care and a mortality rate of up to 4.6%. Treating the infections can cost more than $100,000 per case. The most common bacteria responsible for ODRIs are Staphylococcus (S.) epidermidis and S. aureus, which are responsible for about 70% of all cases. Treatments for ODRIs often include long-term antimicrobial treatment and removal of the implant. Current treatment modalities such as antibiotics, surgical intervention, and/or biofilm disruption have made appreciable strides in infection control, but there still remains a need to reduce or prevent infections associated with biomedical devices, orthopedic implants, and other bodily implants.

One technical problem with antibiotics, for example, is that infectious agents can develop resistance. Further, antibiotics can be coated on the surface of the implant with intermediate agents to cause a bolus release with no long-term elution. For some implants, surface geometries have been used to prevent biofilms and bacterial colonization, but the associated manufacturing methods may also slow osteoblast formation and desired bony ingrowth. The devices, compositions, and methods described herein provide technical solutions to the above identified technical problems. The technical solutions provided herein include using a transition metal porphyrin complex, such as a zinc porphyrin, sometimes abbreviated as ZnPor or ZnPor (II), or a chemical mixture comprising a ZnPor as a coating on the implant and/or eluting a solution of ZnPor from the implant over time. There is no documented infectious resistance to ZnPor, so slow release from an implant over time may reduce or prevent infections associated with the implant. Additionally, or alternatively, ZnPor incorporated into an implant structure (within a reservoir of the implant, coated on the implant, incorporated into the implant material) may cause release from at least a portion of the surface area of the implant. ZnPor has been shown to prevent biofilms, so longer-term elution may reduce or prevent delayed implant associated infections. Additionally, or alternatively, a ZnPor containing implant (within a reservoir of the implant, coated on the implant, incorporated into the implant material) may not need such surface geometry changes, thus obviating issues with osteoblast formation and bony ingrowth.

Porphyrins are tetrapyrroles macrocycles capable of binding metal ions. Porphyrins can exhibit diverse functions in nature as, for example, 1) the heme in hemoglobin, a porphyrin with an Fe ion in the center and 2) the active center of chlorophyll, a Mg-containing porphyrin. The chlorophyll porphyrin can enable the organism to harvest light for the production of energy compounds. In both cases, while different from each other, the porphyrin, or catalytic center, can serve as the active moiety of both heme and chlorophyll molecules. Porphyrins can also be found in cytochromes in bacteria and animals, such as the P450 class of enzymes. In the case of cytochromes, the porphyrin can mediate necessary redox functions in cells that result in the organism producing energy via an electron transport chain.

The porphyrin ZnPor (also described as ZnPor (II) or zinc porphyrin) is water soluble and has a transition metal (II) cation, e.g., zinc (II), inserted into the porphyrin core. The transition metal (II) cation in the porphyrin core increases the excited state lifetime when exposed to light, but these transition metal porphyrin complexes can kill microbes (e.g., virus, bacteria, etc.) in the dark (i.e., the absence of light). For example, ZnPOR has been used to kill Staphylococcus aureus.

The transition metal porphyrin complexes disclosed herein are effective photosensitizers. The transition metal porphyrin complexes when compared to a commercially available photosensitizer, such as Photofrin, has several advantages: (1) it is easier to purify; (2) it need not be a mixture (the transition metal porphyrin is merely present in water); (3) it has a higher binding affinity toward DNA; (4) it is efficient at photoreactions with DNA at low energy; and (4) it is efficient at killing bacteria and viruses in the dark.

The transition metal porphyrins disclosed herein have the general formula (I) shown below.

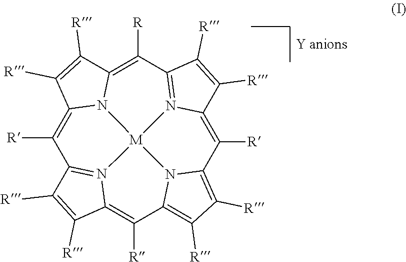

wherein,
M is a transition metal (II) cation,
R is a mono-, di-, tri-, tetra-, or penta-halophenyl and the halogen is selected from the group consisting of Cl, F, Br, and combinations thereof,
R′ is

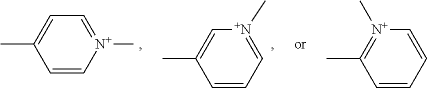

R″ is equal to R or R′,
R′″ is —H, —F, —Cl, or —Br, and
Y is 2 or 3.

In some embodiments, the transition metal M is a Fe⁺², a Co⁺², a Ni⁺², or Zn⁺².

In an embodiment, R″ is equal to R′ and Mis Zn⁺². In this embodiment, R′ may be N-methyl-4-pyridyl, R is pentfluorophenyl, R″ is —H and Y equals 3 and the anion is p-toluene sulfonate. This porphyrin is a meso-5,10,15-tris(N-methyl-4-pyridyl)-20-(pentafluorophenyl) porphyrinatozinc (II), tris-p-toluene sulfonate (ZnPFPTMP) and shown below as formula II.

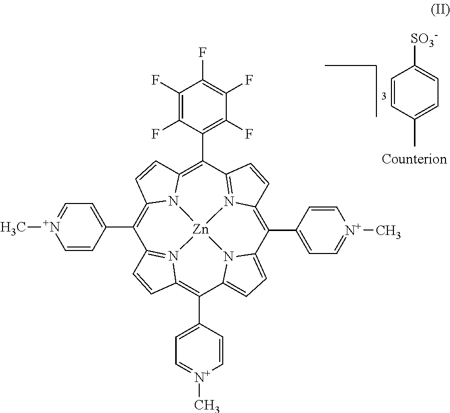

In another embodiment, R″ is equal to R, R is pentfluorophenyl, and Mis Zn⁺². In this embodiment, R′ is N-methyl-4-pyridyl, R″ is —H, Y equals 2 and the anion is p-toluene sulfonate cations. This porphyrin is a meso-5,15-di(N-methyl-4-pyridyl)-10,20-di(pentafluorophenyl) porphyrinatozinc (II), di-p-toluene sulfonate and shown below in formula III.

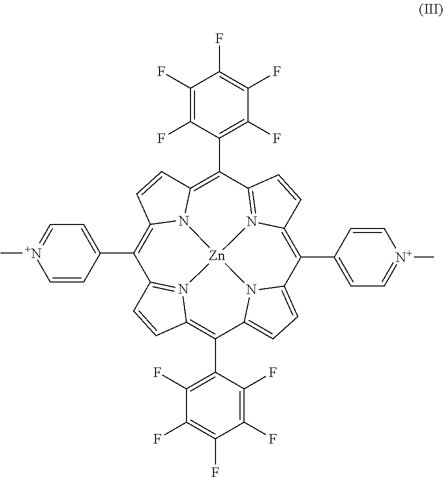

The porphyrins of general formula (I) are water soluble. Further, the porphyrins of general formula (I) kill bacteria and viruses in the dark. The results are shown and described in detail in U.S. Pat. Nos. 8,551,456; 9,364,537; and 11,975,009, the contents of each of which are herein incorporated by reference in their entirety. The porphyrins of formulae (II) and (III) can both be considered embodiments of a ZnPor as described herein.

The ability to kill bacteria in the dark was shown by treating Pseudomonas aeruginosa cells: planktonic and biofilm associated cells with ZnPFPTMP in the absence of light. ZnPFPTMP was added to cell suspensions at the concentrations 0 μM to 100 μM and incubated at 37° C. in the absence of light. At the end of seven (7) hours, viability was determined by plating cell suspensions onto Luria broth (LB) agar plates and determining the viable plate counts (CFU/mL). The MLC (minimum lethal concentration) was determined to be 25 μM, where there were no survivors. There was substantial killing at two (2) hours as well. Biofilms were prevented when the ZnPFPTMP was added to cell suspensions incubated in the dark, presumably due to the toxicity to planktonic cells. When preformed biofilms were exposed to the ZnPFPTMP in the dark there was a 2.7 log reduction in the presence of the Pseudomonas aeruginosa (data not shown).

Additionally, the ability of ZnPFPTMP to affect killing of bacteria in the absence of light was further tested against the Gram-positive pathogenic bacterium Staphylococcus aureus. This bacterium was chosen to evidence the ability of the porphyrins of general formula (I) to treat/kill methicillin-resistant Staphylococcus aureus (MRSA) without exposing the scientists to MRSA directly. Moreover, it is believed that the mechanism used by the porphyrins of general formula (I) is unlikely to involve the methicillin resistance mechanism. S. aureus cells grown in LB medium were washed and re-suspended on 0.2% peptone and exposed to either 1.5 μM or 3.0 μM Zeke in sterile water. Cell suspensions were sampled every 30 minutes for 3 hours and plated to determine viability. After 30 min in the presence of 3.0 μM Zeke, there was a 5.5 log reduction in viable cells of S. aureus, and after 60 min., no viable cells remained. At 1.5 μM, Zeke's complete elimination of viable cells of S. aureus took only two hours.

The ability to kill viruses in the dark was shown by treating Phage PEV2 and a Coronavirus isolated from a cold (ATCC). ZnPor prevented viral infection. ZnPor can interact with many macromolecules and may bind to important components of a virus, rendering it unable to infect cells. A number of studies have shown that light activation of select porphyrins is effective at killing animal viruses. This is due to the high levels of ROSs (Reactive Oxygen Species, e.g., superoxide) produced by activation of the porphyrin and which destroy a wide range of macromolecules (proteins, lipids etc.). In contrast, ZnPor was able to inactivate viruses without light activation. In addition to ZnPor's ability to intercalate into viral RNA and DNA biopolymers, ZnPor can bind to proteins, which may have accounted for how ZnPor acted, in that it bound to the viral proteins such as the spike protein of Coronaviruses. If the Spike proteins bound to ZnPor then the virus couldn't attach to the host cell binding receptor for the spike proteins. There are relatively few antiviral agents. While there are a number of porphyrins that can inactivate viruses using light activation, ZnPor porphyrin has antiviral activity without light activation.

Although the various experimental results described herein use ZnPor, any transition metal porphyrin may be used. For example, light activated porphyrins or porphyrins not needing light activation. In various embodiments, cationic porphyrins may be used.

Cationic porphyrins are known in the art and any of those known are suitable for use herein as a transition metal porphyrin complex, including, but not limited to, transition metal complexed tetra-substituted N-methyl-pyridyl-porphine (TMP), specifically, 5,10,15,20-tetrakis(1-methyl-pyridino)-21H,23H-porphine, including the tetra-p-tosylate salt; 5,10,15,20-tetra(N-methyl-4-pyridiniumyl) porphyrin (TMPyP); 5,10,15,20-tetra-(N-methyl-4-pyridyl) porphine (TMPyP4), and 5,10,15,20-tetra-(N-methyl-2-pyridyl) porphine (TMPyP2). The particular TMP-substituted transition metal prophyrin complex listed above is highly water soluble and effective at low concentrations and does not appear to be toxic to human fibroblasts.

In general, an implant may be an orthopedic implant, a dental implant, bodily implant, or other implantable device. The implant may be formed by sintering, casting, injection molding, or machining. The implant may include or be formed of, at least in part, a biocompatible material. The biocompatible material may include a stainless steel, a titanium alloy, a ceramic, a cobalt chromium alloy, an oxidized zirconium alloy, bone cement, polyethylene, a polymer, or a combination thereof. The implant may be a joint implant (e.g., hip, shoulder, knee, finger, wrist ankle, foot, etc.), a bone plate, an intramedullary rod, a bone graft, a spinal implant, a dental implant, or other implantable device.

An infection control (e.g., infection prevention, infection reducing, etc.) composition may include a porphyrin, for example, as a porphyrin solution, a porphyrin powder, etc. An infection control composition may include a porphyrin and one or more pharmaceutically acceptable excipients as described herein. A “pharmaceutically acceptable” excipient can refer to a component that is not biologically or otherwise undesirable, e.g., the excipient is useful in preparing a pharmaceutical or therapeutic composition that is generally safe and non-toxic, acceptable for veterinary and/or human pharmaceutical or therapeutic use, and may be incorporated into a pharmaceutical formulation (e.g., an infection control composition) provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

The terms “excipient” or “pharmaceutically acceptable excipient” can include, but are not limited to, solvents, suspensions, solutions, powders, and polymers such as phosphate buffered saline solution, water, and emulsions (such as an oil/water or water/oil emulsion), as well as diluents, surfactants, thickeners, fillers, salts, buffers, stabilizers, solubilizers, lipids, preservatives, disinfectants, wetting agents, or other material well known in the art for use in pharmaceutical formulations and as described further herein.

A weight ratio of the porphyrin to the pharmaceutically acceptable excipient may be about 1:1000 to about 1:500,000; about 1:2000 to about 1:500,000; about 1:5000 to about 1:500,000; about 1:2,000 to about 1:500,000; about 1:10,000 to about 1:500,000; about 1:2,000 to about 1:250,000; about 1:10,000 to about 1:250,000; about 1:2,000 to about 1:125,000; about 1:10,000 to about 1:125,000; about 1:10,000 to about 1:75,000; about 1:10,000 to about 1:50,000; about 1:12,500 to about 1:125,000; about 1:25,000 to about 1:100,000; about 1:50,000 to about 1:75,000; about 1:50,000 to about 1:100,000; or about 1:50,000 to about 1:125,000. For example, a weight ratio of the porphyrin to the pharmaceutically acceptable excipient may be about 1:1000 to about 1:500,000. For example, a weight ratio of the porphyrin to the pharmaceutically acceptable excipient may be about 1:50,000 to about 1:75,000.

An effective concentration of the porphyrin may be about 1 μM to about 1000 μM; 1 μM to about 500 μM; 1 μM to about 325 μM; 1 μM to about 100 μM; about 3 μM to about 100 μM; about 3 μM to about 325 μM; about 3 μM to about 500 μM; about 5 μM to about 100 μM; about 10 μM to about 50 μM; about 25 μM to about 75 μM; about 50 M to about 100 μM; about 75 μM to about 100 μM; about 5 μM to about 15 M; about 10 μM to about 20 μM; about 10 μM to about 40 μM; about 60 μM to about 80 M; about 80 μM to about 100 M; about 80 μM to about 325 μM; or about 80 μM to about 500 μM. For example, an effective concentration of the porphyrin may be about 1 μM to about 1000 μM. For example, an effective concentration of the porphyrin may be about 80 M to about 100 μM.

An effective concentration of the porphyrin may be about 2 μg/mL to about 1000 μg/mL; about 2 μg/mL to about 750 μg/mL; about 2 μg/mL to about 500 μg/mL; 2 μg/mL to about 150 μg/mL; about 4 μg/mL to about 500 μg/mL; about 4 μg/mL to about 100 μg/mL; about 10 μg/mL to about 75 μg/mL; about 12.5 μg/mL to about 50 μg/mL; about 25 μg/mL to about 50 μg/mL; about 10 μg/mL to about 100 μg/mL; about 25 μg/mL to about 100 μg/mL; about 25 μg/mL to about 75 μg/mL; about 50 g/mL to about 100 μg/mL; about 75 μg/mL to about 100 μg/mL; about 50 g/mL to about 150 μg/mL; about 50 μg/mL to about 500 μg/mL; about 50 μg/mL to about 1000 μg/mL; about 2 μg/mL to about 50 μg/mL; about 4 μg/mL to about 75 μg/mL; or about 4 μg/mL to about 50 μg/mL. For example, an effective concentration of the porphyrin may be about 2 μg/mL to about 1000 μg/mL. For example, an effective concentration of the porphyrin may be about 4 μg/mL to about 50 g/mL.

The pharmaceutically acceptable excipient may include, but is not limited to, saline, water, a disinfectant, or a combination thereof. In some embodiments, the infection control composition may include one or more elements that increase surface application, for example one or more of water, glycerol, ethanol, propanediol, butylene glycol, dipropylene glycol, ethoxylated or propoxylated diglycols, cyclic polyols, and the like. For example, the composition may include water to increase surface application. For example, the composition may include glycerol to increase surface application. For example, the composition may include ethanol to increase surface application.

The infection control composition may optionally, additionally, include an antibiotic, as a combination therapy. The combined effect of a porphyrin and the antibiotic is significantly higher than either alone. Further, bacteria are unlikely to develop resistance to the porphyrin as its mechanism of action is to act outside of the cell on the DNA in the extracellular matrix of a biofilm, also known as eDNA, or to generate singlet oxygen which targets the cellular components. In particular, the mechanism of porphyrins can be different from traditional antibiotics: (1) photoactivation generates singlet oxygen which destroys most biomolecules very rapidly, and (2) without photoactivation, the porphyrin intercalates into extracellular DNA and it is unlikely that microbes would modify the structure of their eDNA to avoid this-indeed there is no evidence that organisms have developed a way to avoid intercalating agents from intercalating into their eDNA.

The infection control composition may be applied (e.g., sprayed, coated, soaked, etc.) to at least a portion of the surface of an implant, integrated into a material of the implant (e.g., during manufacturing), and/or at least partially contained within or retained in the implant (e.g., within a reservoir of the implant). Alternatively, or additionally, any of the implants described herein may be manufactured such that the porphyrin is embedded or impregnated in the material of the implants.

A portion of the implant or an external surface of the implant may be substantially resistant to biofilm formation for about one week to about six months. In some embodiments, a portion of the implant or an external surface of the implant may be substantially resistant to biofilm for about two weeks to about three months. In some embodiments, a portion of the implant or an external surface of the implant may be substantially resistant to biofilm for about one month to about two months. In some embodiments, however, the concentration of the infection control composition is sufficiently strong to completely kill off lingering bacteria and other microbes introduced during surgery, therefore fully preventing the formation of a biofilm within the body. Biofilms are surface-associated microbial structures comprising a single microbe (a pure culture), or heterogenous structures comprising different populations of microorganisms, both of which are surrounded by a self-produced matrix that allows for their attachment to animate or inanimate surfaces. Biofilms can include mechanisms of bacterial protection and tissue damage. These can include decreased susceptibility to phagocytosis and strengthened antibiotic resistance due to reduced antibiotic penetration and altered bacterial metabolism. In addition, biofilms can be characterized by continued inflammation, which is dominated by antibody responses and polymorphonuclear leukocytes. Biofilm-associated inflammation can destroy surrounding tissue.

In general, the compositions, devices, and methods described herein function to reduce infection or biofilm formation on or associated with an implant.

In general, the compositions, devices, and methods described herein function to reduce or prevent infection, due to microorganisms, on or associated with an implant.

In general, the compositions, devices, and methods described herein have antimicrobial activity, antibacterial activity, or antiviral activity.

As used herein, “microorganisms” include, but are not limited to, bacteria, viruses, fungi, algae, yeasts, protozoa, spirochetes, single-celled and multi-celled organisms that are included in classification schema as prokaryotes, eukaryotes, Archaea, Bacteria and those that are known to those skilled in the art. Microorganisms may also refer to isolated microorganisms and may comprise particular deposited compositions or microorganisms disclosed herein, and the intent of the text can be interpreted by those of skill in the art.

For example, the devices, compositions, and methods described herein may be capable of reducing or preventing infection, associated or on an implant, due to gram positive bacteria, gram negative bacteria, and/or acid-fast bacteria.

Non-limiting examples of bacteria can include Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium smegmatis, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumonias, Listeria monocytogenes, Listeria ivanovii, Bacillus subtilis, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus aureus MRSA and MSSA strains, Staphylococcus epidermidis, Streptococcus mutans, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, Pseudomonas aeruginosa PA14, Pseudomonas aeruginosa PAO1, other Pseudomonas species, Haemophilus influenzae, Haemophilus ducreyi, other Hemophilus species, Clostridium tetani, other Clostridium species, Yersinia enterolitica, and other Yersinia species, or any combination thereof.

For example, the devices, compositions, and methods described herein may be capable of reducing or preventing infection, on or associated with an implant, due to viruses.

Non-limiting examples of viruses include Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (such as, for example, avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV) porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV) (SARS-COV), Severe Acute Respiratory Syndrome (SARS)-Coronavirus (CoV)-2 (SARS-CoV-2) (including, but not limited to the B1.351 variant, B.1.1.7 variant, USA-WA1/2020, or P.1 variant), or middle east respiratory syndrome (MERS) coronavirus (CoV) (MERS-COV)), Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

For example, the devices, compositions, and methods described herein may be capable of reducing or preventing infection, on or associated with an implant, due to fungi.

Nonlimiting examples of fungi can include Candida albicans, Cryptococcus neoformans, Histoplasma capsulatum, Aspergillus fumigatus, Coccidioides immitis, Paracoccidioides brasiliensis, Blastomyces dermitidis, Pneumocystis carnii, Penicillium marneffi, and Alternaria alternata.

In some embodiments, a period of time of infection control may span about 1 month to about 10 years; about 1 month to about 3 months; about 3 months to about 6 months; about 6 months to about one year; about one year to about five years; about 5 years to about 10 years; etc. In some embodiments, a period of time of infection control may span for greater than about one month; greater than about 3 months; greater than about 6 months; greater than about one year; or greater than about 5 years. In some embodiments, a period of time of infection control may span for at least about one month; at least about 3 months; at least about 6 months; at least about one year; or at least about 5 years.

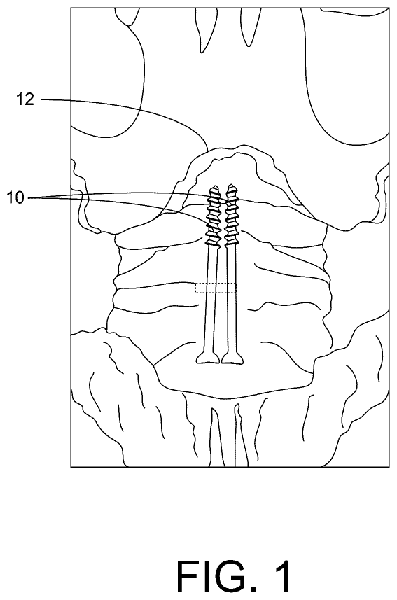

FIG. 1 shows a schematic of an x-ray image of an embodiment of a bone screw 10 anchored in a bone 12. A bone screw is an implant used in orthopedic and trauma surgery to secure bone fragments together or to attach implants (such as plates or rods) to bones. The bone screw 10 may be surface treated with an infection control composition including one or more porphyrins, for example ZnPor. The bone screw 10 may be sprayed with an infection control composition. The bone screw 10 may be soaked in an infection control composition. The bone screw 10 may define a reservoir (not shown) within a body of the bone screw 10 that contains an infection control composition. The bone screw 10 may be manufactured such that the porphyrin is embedded or impregnated in the material of the bone screw 10. The infection control composition may elute from the surface, the reservoir, or the material of the bone screw over time. The bone screw 10 may be pretreated with the infection control composition prior to implantation in the bone 12. The bone screw 10 may be implanted in the bone 12, and the reservoir filled with (prior to bone screw implantation or after bone screw implantation) the infection control composition. The bone screw 10 may be sprayed with the infection control composition and implanted in the bone 12.

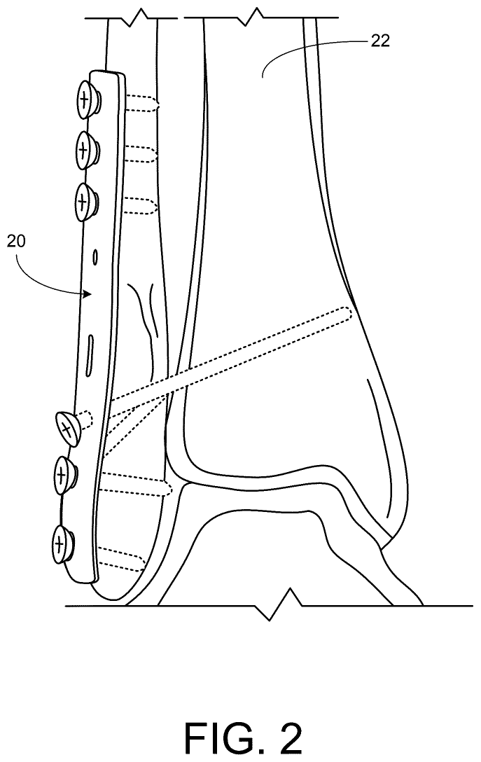

FIG. 2 shows a schematic of an x-ray image of an embodiment of a bone plate 20 anchored to a bone 22. A bone plate 20 is an orthopedic implant used in the treatment of fractures, particularly in cases where the fracture is not amenable to intramedullary nailing or other forms of fixation. A purpose of a bone plate 20 is to provide stabilization and support to fractured bones during the healing process. The bone plate 20 may be surface treated with an infection control composition including one or more porphyrins, for example ZnPor. The bone plate 20 may be sprayed with an infection control composition. The bone plate 20 may be soaked in an infection control composition. The bone plate 20 may define a reservoir (not shown) within a body of the bone plate 20 that contains an infection control composition. The bone plate 20 may be manufactured such that the porphyrin is embedded or impregnated in the material of the bone plate 20. The infection control composition may elute from the surface, the reservoir, or the material of the bone plate over time. The bone plate 20 may be pretreated with the infection control composition prior to implantation in the bone 22. The bone plate 20 may be implanted in the bone 22, and the reservoir filled with (prior to implantation or after implantation) the infection control composition. The bone plate 20 may be implanted in the bone 22, and the bone plate 20 sprayed with the infection control composition after implantation.

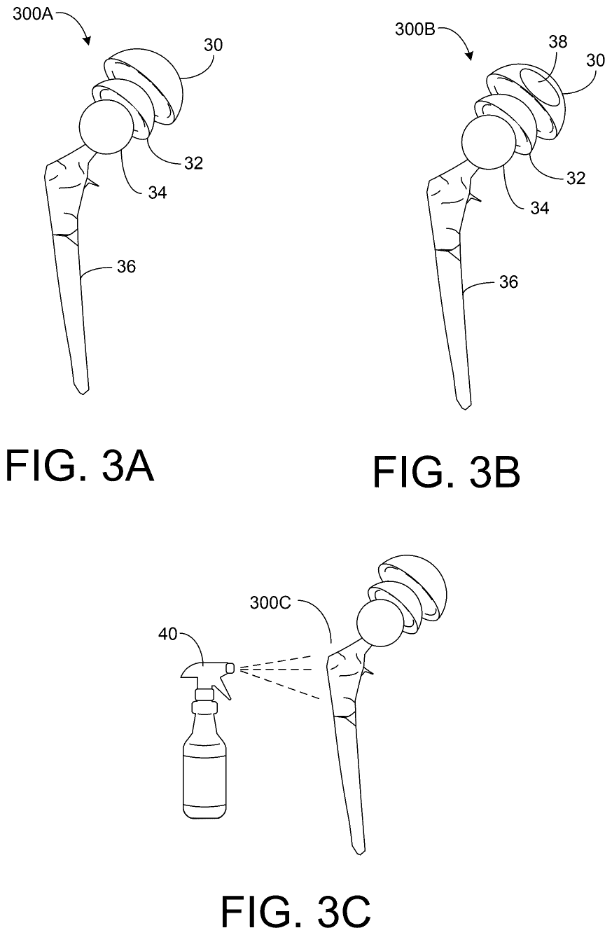

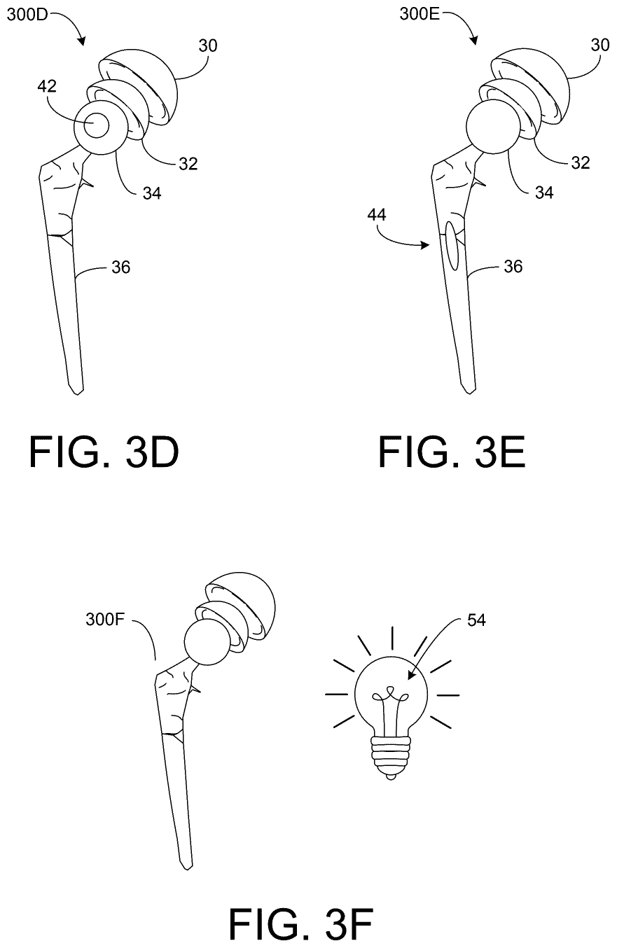

FIGS. 3A-3F show various embodiments of an orthopedic implant 300A, 300B, 300C, 300D, 300E, 300F. The implants 300A, 300B, 300C, 300D, 300E, 300F include an acetabular component 30, a liner 32, a femoral head 34, and a stem 36. As shown in FIG. 3A, the implant 300A may be surface treated with an infection control composition including one or more porphyrins, for example ZnPor. The implants 300A may be soaked in an infection control composition.

As shown in FIGS. 3B, 3D, 3E, the implants 300B, 300D, 300E, respectively, may define a reservoir 38, 42, 44, respectively, within a body of the implant 300B, 300D, 300E that contains an infection control composition. The one or more reservoirs may be sized, shaped, and/or structured to elute, leach, or discharge the porphyrin composition, solution, powder, or the like over time. In some embodiments, the reservoir can be defined by one or more sidewalls defining an empty volume within. In some embodiments, one or more channels can direct the porphyrin composition from the reservoir to a surface of the implant and into the surrounding tissue.

As shown in FIG. 3B, the reservoir 38 may be defined by the acetabular component 30 of the implant 300B. As shown in FIG. 3D, the reservoir 42 may be defined by the femoral head 34 of the implant 300D. As shown in FIG. 3E, the reservoir 44 may be defined by the stem 36 of the implant 300E. In some embodiments, the implant may define one or more reservoirs or a plurality of reservoirs. For example, the implant may define one or more reservoirs in the acetabular component 30 and the stem 36. The implant may define one or more reservoirs in the stem 36 and the femoral head 34. The implant may define one or more reservoirs in the femoral head 34 and the acetabular component 30. The implant may define one or more reservoirs in the femoral head 34, acetabular component 30, and the stem 36. The implant may additionally, or alternatively, define a reservoir in a liner 32 of the implant. The reservoir in the liner 32 may optionally be combined with any one or more of a reservoir in the stem 36, femoral head 34, or acetabular component 30. A volume of a reservoir of the one or more reservoirs defined by the implant body may be about 1 mL to about 10 mL, in some embodiments. In other embodiments, the volume may be about 1 mL to about 7.5 mL, about 1 mL to about 5 mL, about 1 mL to about 2.5 mL, about 5 mL to about 10 mL, and about 7.5 mL to about 10 mL. For example, a volume of the reservoir defined by the implant body may be about 1 mL to about 10 mL. For example, a volume of the reservoir defined by the implant body may be about 1 mL to about 2.5 mL. A concentration of the porphyrin in the infection control solution in the reservoir may be about 2 μg/mL to about 1000 μg/mL, in some embodiments. In some embodiments, a concentration of the porphyrin may be about 4 μg/mL to about 500 μg/mL. In some embodiments, a concentration of the porphyrin may be about 4 μg/mL to about 150 μg/mL. In some embodiments, a concentration of the porphyrin may be about 4 μg/mL to about 100 μg/mL. In some embodiments, a concentration of the porphyrin may be about 4 μg/mL to about 75 g/mL. In some embodiments, a concentration of porphyrin may be about 4 μg/mL to about 50 μg/mL. In some embodiments, a concentration of porphyrin may be about 25 μg/mL to about 75 μg/mL.

In some embodiments, a portion of the implant or a body of the implant may be porous, such that the infection control composition may elute from the reservoir and through the body of the implant into a surrounding tissue. A porosity of the implant body may be about 30% to about 80%; about 30% to about 40%; about 40% to about 50%; about 50% to about 60%; about 60% to about 70%; or about 70% to about 80%.

The implants 300B, 300D, 300E may be implanted, and the reservoir filled with the infection control composition. Alternatively, the implants 300B, 300D, 300E may be pre-filled with the infection control composition or filled just prior to implantation.

As shown in FIG. 3C, the implant 300C may be sprayed, using applicator 40, with an infection control composition. The implant 300C may be sprayed before implantation or after implantation. The implant 300C may be incubated for a predefined time period with the sprayed composition thereon and then implanted, or excess composition may be removed before implantation. For example, the predefined time period may be about 1 minute to about 1 hour, about 1 minute to about 45 minutes, about 1 minute to about 30 minutes, about 1 minute to about 20 minutes, about 20 minutes to about 1 hour, about 30 minutes to about 1 hour, and about 45 minutes to about 1 hour.

FIG. 3F shows an embodiment of an orthopedic implant 300F being treated with a light-activated infection control composition. The implant 300F may be pre-treated with, soaked in, sprayed with, or otherwise treated with a light-activated porphyrin. The implant may be light activated, using a light source 54, before implantation or after implantation. The light activation may occur for a predefined period of time, for example about 1 minute to about 2 hours, about 1 minute to about 1 hour, about 1 minute to about 45 minutes, about 1 minute to about 30 minutes, and about 1 minute to about 15 minutes. The light may have a wavelength of about 400 nm to about 850 nm. In some embodiments, the light source 54 may be a visible light source or a light emitting diode (LED). The visible light can be provided with low power lasers. The low power of the visible light may be from about 200 W to about 400 W, about 250 W to about 350 W, about 275 W to about 325 W, or about 290 W to about 310 W.

The infection control composition may elute from the surface, the reservoir, or the material of the implant over time. Any of the implants 300A, 300B, 300C, 300D, 300E, 300F may be pretreated with the infection control composition prior to implantation.

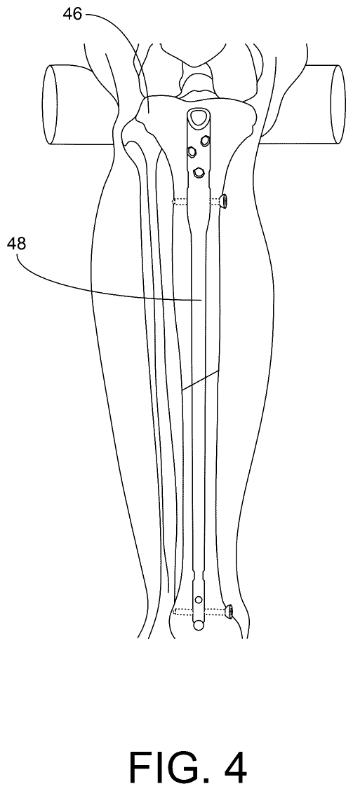

FIG. 4 shows a side view of an embodiment of an intramedullary nail 48 implanted in a bone 46. An intramedullary nail 48 is a type of orthopedic implant used in the treatment of fractures of long bones, such as the femur and tibia. An intramedullary nail provides stability and support to fractured bones during the healing process. The intramedullary nail 48 may be surface treated with an infection control composition including one or more porphyrins, for example ZnPor. The intramedullary nail 48 may be sprayed with an infection control composition. The intramedullary nail 48 may be soaked in an infection control composition. The intramedullary nail 48 may define a reservoir (not shown) within a body of the intramedullary nail 48 that contains an infection control composition. The intramedullary nail 48 may be manufactured such that the porphyrin is embedded or impregnated in the material of the intramedullary nail 48. The infection control composition may elute from the surface, the reservoir, or the material of the intramedullary nail 48 over time. The intramedullary nail 48 may be pretreated with the infection control composition prior to implantation in the bone 46. The intramedullary nail 48 may be implanted in the bone 46, and the reservoir filled with the infection control composition. The intramedullary nail 48 may be sprayed with the infection control composition and implanted in the bone 46.

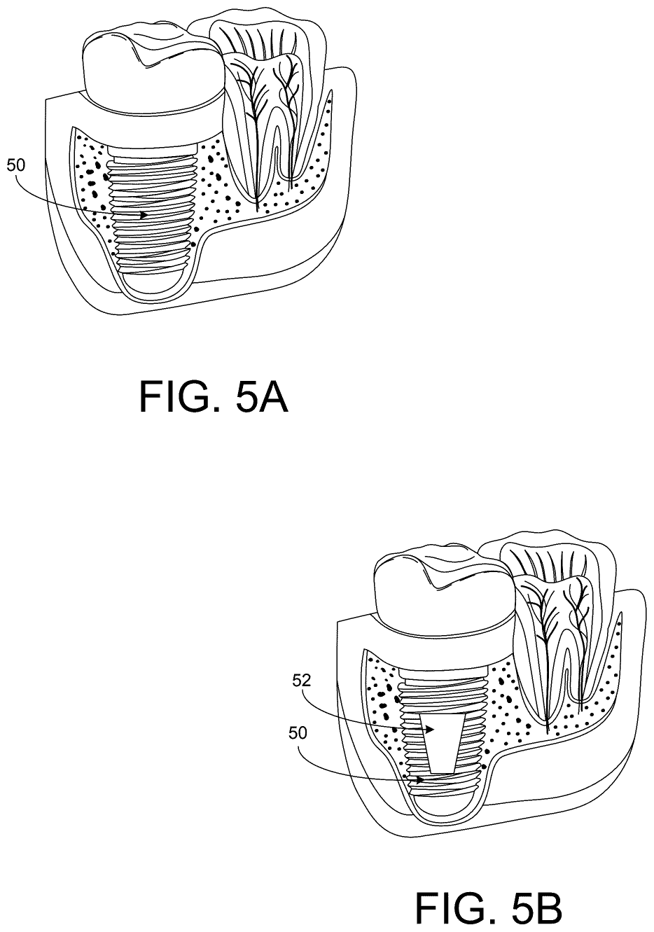

FIG. 5A shows a perspective view of a dental implant 50. A dental implant 50 serves as a replacement for a missing tooth root and provides a foundation for a prosthetic tooth or teeth. The dental implant 50 may be surface treated with an infection control composition including one or more porphyrins, for example ZnPor. The dental implant 50 may be sprayed with an infection control composition. The dental implant 50 may be soaked in an infection control composition. The dental implant 50 may be manufactured such that the porphyrin is embedded or impregnated in the material of the dental implant 50. The dental implant 50 may be pretreated with the infection control composition prior to implantation in the bone. The dental implant 50 may be implanted in the bone 46, and the implant sprayed with the infection control composition after implantation.

FIG. 5B shows a perspective view of a dental implant 50 defining a reservoir 52. The dental implant 50 may define a reservoir 52, within a body of the dental implant 50, that contains an infection control composition. The dental implant 50 may be implanted in the bone, and the reservoir 52 filled with the infection control composition. The dental implant 50 may be pre-filled (e.g., during manufacturing) with the infection control composition or filled just prior to implantation. The infection control composition may elute from the surface of the dental implant 50, the reservoir, or the material of the dental implant 50 over time.

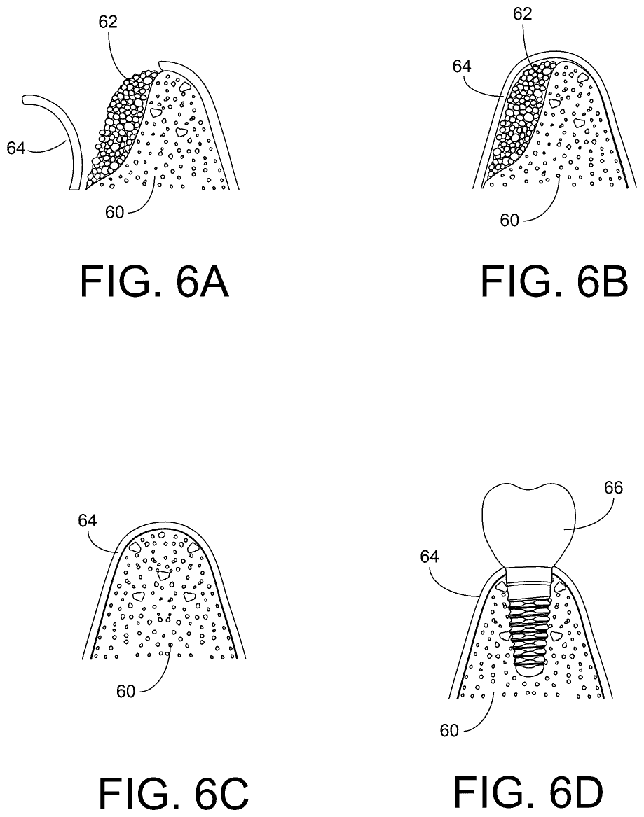

FIGS. 6A-6D show an embodiment of a method of applying bone graft material 62, including an infection control composition, to a bone 60 (e.g., jawbone). Bone grafting involves the transplantation or placement of bone tissue to repair or regenerate bone that has been lost or damaged. This procedure may be used in dentistry to support dental implants, treat jawbone defects, or enhance bone structure for various oral and maxillofacial procedures. More generally, bone grafting may be used to repair, restore, or regenerate bone that has been lost or damaged due to injury, disease, or other conditions. As shown in FIG. 6A, bone graft material 62 may be added to or applied to the recess in the bone 60 after soft tissue 64 has been peeled back to expose the recess. The bone graft material 62 may be autograft, allograft, or xenograft homogenized bone that includes an infection control composition (e.g., solution, powder, etc.) mixed in with the homogenized bone. The bone graft material 62 may be a synthetic material (e.g., hydroxyapatite, tricalcium phosphate, or bioactive glass) that includes an infection control composition, solution, powder, etc. mixed in with the synthetic material. The bone graft material 62 may be demineralized bone matrix that includes an infection control composition (e.g., solution, powder, etc.) mixed in with the demineralized bone matrix. The bone graft material 62 may be a composite graft that includes an infection control composition (e.g., solution, powder, etc.) mixed in with the composite graft. In some embodiments, various growth factors (e.g., bone morphogenetic protein (BMP), platelet-derived growth factor (PDGF), vascular endothelial growth factor (VEGF), etc.) may be added to the bone graft material 62. As shown in FIG. 6B, tissue may be positioned to overlay the bone graft material 62. Once healing is complete or substantially complete, as shown in FIG. 6C, the graft site may be used for an implant 66, for example a dental implant, as shown in FIG. 6D.

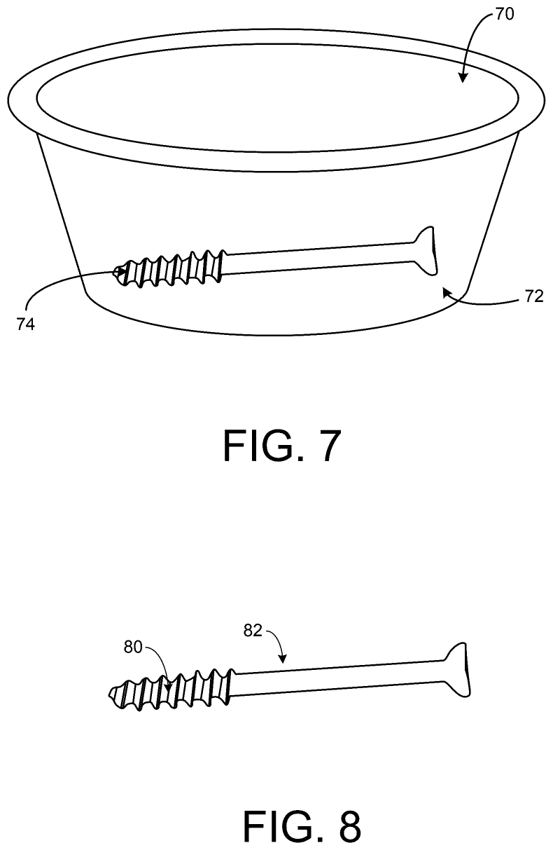

FIG. 7 shows a receptacle 70 sized and shaped to receive an implant 74 therein for applying an infection control composition 72 to the implant 74. The implant 74 may be soaked in the infection control composition 72 for a predefined time period, for example about 1 minute to about 1 hour, about 1 minute to about 45 minutes, about 1 minute to about 30 minutes, about 1 minute to about 20 minutes, about 20 minutes to about 1 hour, about 30 minutes to about 1 hour, and about 45 minutes to about 1 hour. The implant 74 may be directly implanted at a target site after the soaking or may be at least partially dried (e.g., air dried, oven dried, etc.) before implantation at a target site.

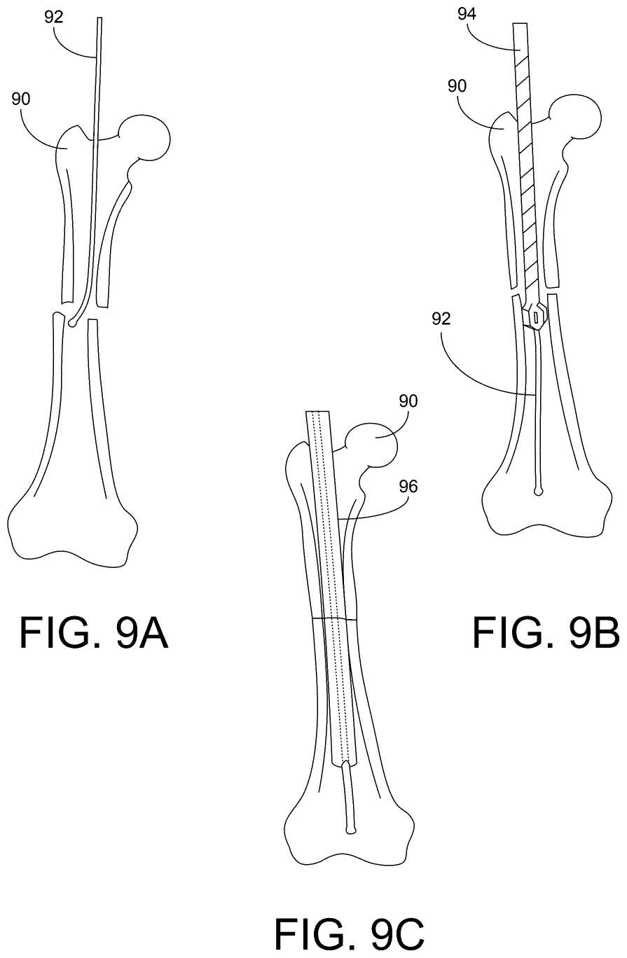

FIG. 8 shows an embodiment of an implant 80 having an infection control coating 82. The infection control coating 82 may include hydroxyapatite, a porphyrin, ZnPor, or a combination thereof. In some embodiments, ZnPor may bind to a hydroxyapatite-coated surface. For example, an implant body formed of a biocompatible material may include a hydroxyapatite coating on at least a portion of an exterior surface of the implant body. The infection control composition (e.g., porphyrin composition, solution, etc.) may react with or bond to the hydroxyapatite coating,

FIGS. 9A-9C shows an embodiment of a method for applying an infection control composition to a bone reaming process and/or bone reamings. Bone reaming is a surgical procedure performed to prepare the intramedullary canal for the insertion of an orthopedic implant, such as intramedullary nail used in the fixation of fractures. As shown in FIG. 9A, a ball tipped guidewire 92 may be inserted into the intramedullary canal 96 of the bone 90. As shown in FIG. 9B, a reamer 94 may be passed over the guidewire 92 in the intramedullary canal 96 to ream the canal 96. In some embodiments, as shown in FIG. 9C, a series of progressively larger reamers may be inserted over the guidewire 92 to enlarge and shape the canal 96. Once reaming is complete, the guidewire 92 and reamer 94 may be removed and an implant 96 may be inserted into the intramedullary canal 96. An infection control composition (e.g., solution, poweder, etc.) may be applied to the reamed intramedullary canal during or after reaming and/or applied to the reamings that are remaining in the canal 96 from the reaming procedure.

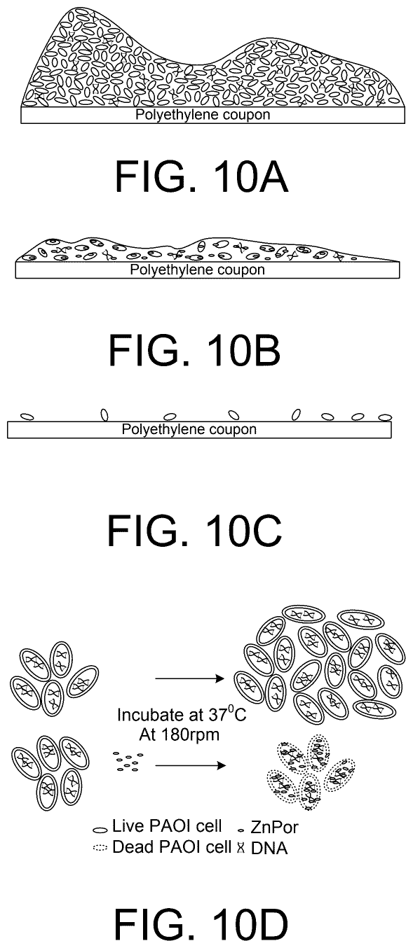

FIGS. 10A-10D show a proposed model of the mechanism of action of ZnPor against PsA biofilms and individual cells. FIG. 10A represents a PsA biofilm 16 hours to 18 hours after treatment with a LIVE/DEAD™ stain. The biofilm is a thick layer of cells encased in an extracellular matrix (ECM). All or almost all cells are green from the LIVE/DEAD™ stain. The ECM contains a variety of different biomolecules e.g., eDNA, which constitutes the majority of the ECM. ZnPor can diffuse throughout the ECM (as shown in FIG. 10B) and thus interact with the eDNA in the matrix. FIGS. 10B-10C show that ZnPor treatment (prior to LIVE/DEAD™ staining) has the effect of destabilizing the biofilm, and ultimately the biofilm sloughs off the surface (FIGS. 10B-10C). The LIVE/DEAD™ stained biofilms depict dead biofilm-associated cells left on the surface after ZnPor treatment. Overall, the matrix of biofilms treated with ZnPor are converted from a thick and dense matrix to a thin monolayer of almost exclusively dead cells and detachment of the biofilm from the surface. FIG. 10D shows individual planktonic PsA cells rapidly accumulate ZnPor in the cytoplasm and membrane/cell wall. Porphyrins bind to DNA either intercalating and/or binding to the outside of the helix. ZnPor treated cells do not increase in total number (nor do they decrease). These cells do not form colonies when plated onto liquid broth agar plates and thus are not viable. Yet the cells do not lyse. The intact dead cells can be handled by the immune system which may be better than lysing the cells due to the release of endotoxin.

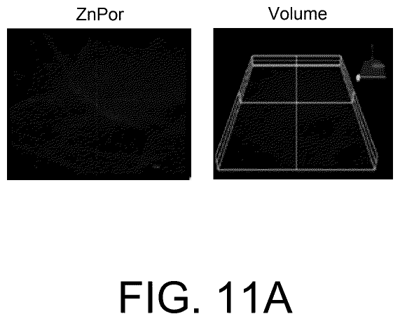

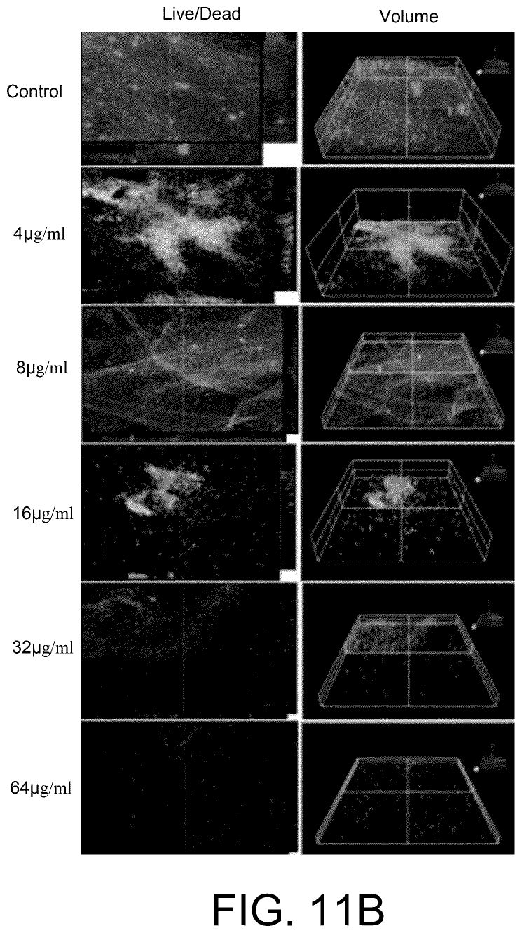

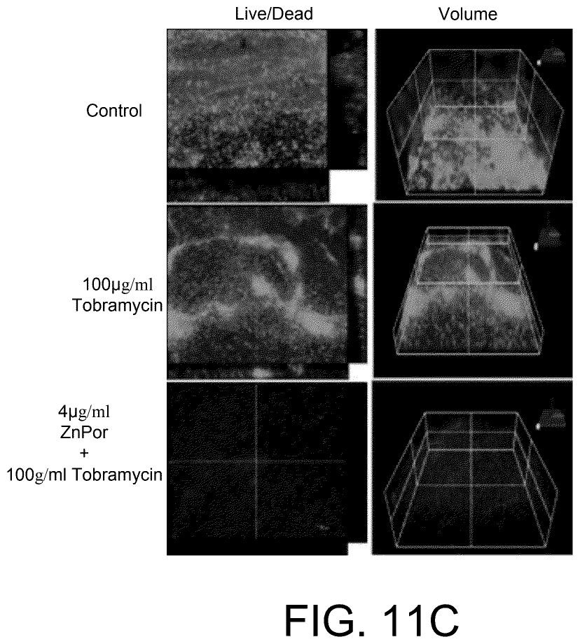

FIGS. 11A-11C show experimental data of the distribution and effect of ZnPor against PsA PAO1 biofilm. FIGS. 11A-11C show confocal microscopy images of PsA biofilms formed on polyethylene coupons in CDC approved bioreactors for 16 hours to 18 hours. FIG. 11A shows ZnPor (alone) stained biofilm. The biofilm was incubated in 8 μg/mL ZnPor solution for 2 h. The distribution of ZnPor in the biofilm was imaged by ZnPor excitation/emission (433 nm/620 nm, respectively). The 3D volumetric depth image shows ZnPor is distributed throughout the biofilm matrix. FIG. 11B shows a LIVE/DEAD™ stain and 3D volumetric depths of biofilms treated with concentrations of ZnPor from 4 μg/mL to 64 μg/mL for 2 hours followed by LIVE/DEAD™ staining. The LIVE/DEAD™ staining shows that biofilm inhibition begins to occur around at least 4 μg/mL evidenced by the increase in yellow staining or dead cells and decrease in green staining or live cells. FIG. 11C shows a LIVE/DEAD™ stain of biofilm treated with 100 μg/mL of Tobramycin alone, or in combination with ZnPor (30 minutes pre-treatment (4 μg/mL)] for 2 hours). Control refers to a biofilm that received no treatment. Tobramycin alone does not affect biofilm formation. Addition of 4 μg/mL ZnPor results in biofilm inhibition. Green means LIVE; red means DEAD. All graphs are representative of three independent experiments (n=9). Length of size bar: 10 μM.

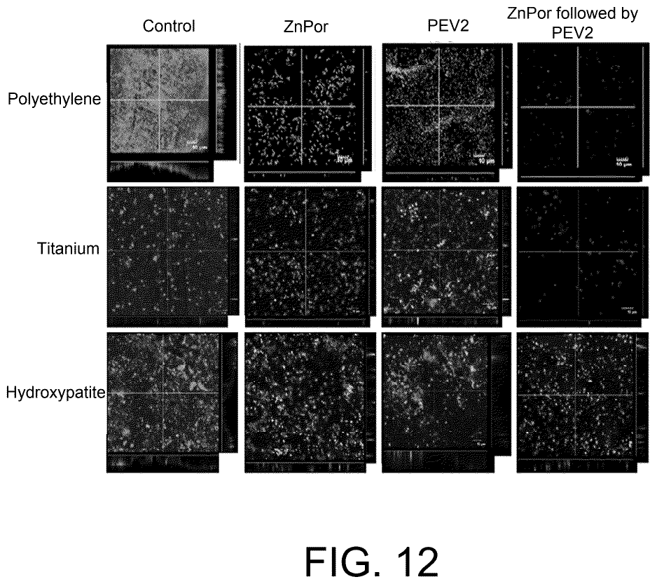

FIG. 12 shows experimental data of PsA PAO1 biofilms treated with ZnPor and PEV2 alone and in combination without photoactivation. Biofilms were grown on coupons of polyethylene, titanium, and hydroxyapatite in CDC approved bioreactors. PsA PAO1 biofilms were grown on coupons in MSG media for about 16 hours to 18 hours. Overnight media was replaced with fresh PBS and biofilms were treated with Column 1: Control; Column 2: ZnPor alone (33 μg/mL) which was incubated for two hours; Column 3: PEV2 alone (multiplicity of infection or MOI of 10:1), which was incubated for four hours; or Column 4: ZnPor at 33 μg/mL (incubated for two hours) followed by the addition of PEV2 at an MOI of 10:1 (incubated for an additional four hours). Coupons were gently washed in distilled water and were treated with LIVE/DEAD™ stain. Coupons were imaged using confocal laser scanning microscopy (CLSM) at 60×. Biofilms treated with ZnPor show a significant reduction in biofilm biomass compared the control. The majority of the cells is dead, and the cells left on the surface present as a monolayer. In the PEV2 treatment, there is approximately a 50% reduction in biofilm biomass compared to the control. Treatments of combined ZnPor and PEV2 show the greatest synergy and the largest reduction in biofilm cells. Green means LIVE; red means DEAD. Control refers to a biofilm that received no treatment of either ZnPor or PEV2. Pictures are representative of total treatments. As shown in FIG. 12, ZnPOR was effective on biocompatible materials, such as polyethylene, titanium, and hydroxyapatite, that may be used in implants, as also described with respect to FIG. 8. Said another way, ZnPor substantially reduces biofilms on hydroxyapatite and kills greater than 95% or greater than 99% of cells.

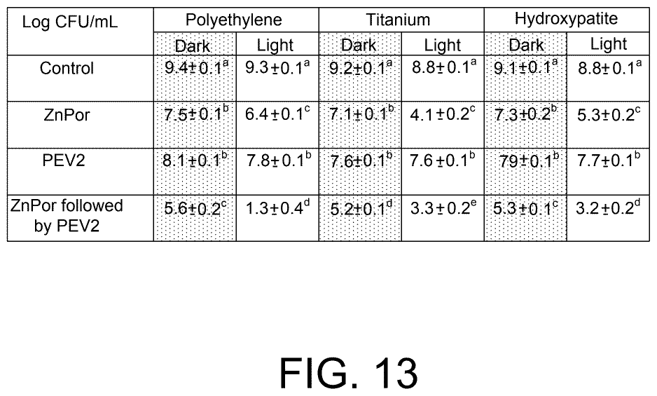

FIG. 13 shows experimental data in table format of PAO1 Biofilms treated with ZnPor in the presence and absence of photoactivation. Viable cell counts (CFU/mL) of biofilms designated for quantitative measurement were determined through serial dilution of biofilm vortexed off coupon. Biofilms were either kept in the dark or photoactivated with a 300 W light with a 420 nm filter for 20 minutes. Each material responds to combinatorial treatment in a similar manner. ZnPor decreases the bacterial titer significantly compared to both the control and PEV2 treatment. However, when photoactivated there was a 5 log reduction in CFU/mL on Titanium coupons. This was not unexpected as ZnPor has greater bactericidal activity when activated with light. Data represents total mean #standard error of the mean (SEM), Log CFU/mL total (n=15, n=18, n=21 respectively). Statistical significance was annotated by the use of letters, a change in letter indicates significance from respective treatments on the same material with a P value of at least 0.05.

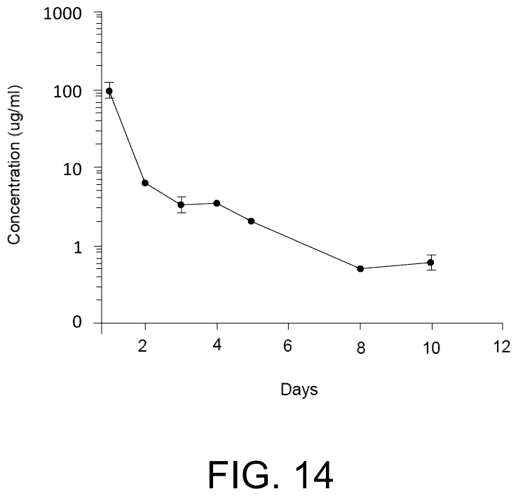

FIG. 14 shows prophetic elution profile data of ZnPor concentration over time. ZnPor may elute from an implant (e.g., reservoir in an implant). When starting at a concentration of about 100 μg/mL, ZnPor may elute over several days (e.g., 10 days) to a concentration of about 1 μg/mL. ZnPor concentration may decrease by about 10-fold within the first about 2-3 days post implantation. A second 10-fold decrease may occur between about day 3 and about day 10. ZnPor may further reduce in concentration after about 10 days.

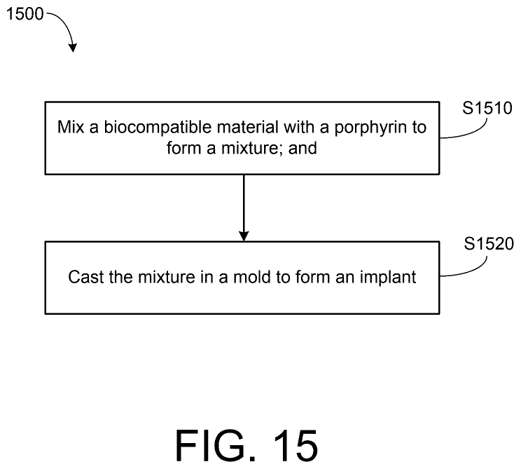

FIG. 15 shows an embodiment of a method 1500 of casting an infection resistant implant. The method 1500 for casting an infection resistant implant of an embodiment includes mixing a biocompatible material with a porphyrin to form a mixture at block S1510; and casting the mixture in a mold to form an implant at block S1520. Implant casting is a manufacturing process used to create metal components, including medical implants. Method 1500 may be used to create orthopedic implants (e.g., hip, knee, shoulder), bone plates, bone screws, dental implants (e.g., dental crowns, bridges, other prosthetic components), cardiovascular devices (e.g., stents, heart valve components, other cardiovascular implants), and/or custom implants. Casting is suitable for patient-specific implants that require bespoke design and precision.

As shown in FIG. 15, an embodiment of a method 1500 of casting an infection resistant implant includes block S1510, which recites mixing a biocompatible material with a porphyrin to form a mixture. For example, molten biocompatible material (e.g., metal) may be mixed with one or more porphyrins, for example ZnPor. A temperature of the molten biocompatible material may be within a predefined temperature range (e.g., greater than about 200 degrees Celcius or between about 200 degrees Celcius and about 400 degrees Celcius) to prevent inactivation or destruction of the porphyrin molecules.

As shown in FIG. 15, an embodiment of a method 1500 of casting an infection resistant implant includes block S1520, which recites casting the mixture in a mold to form an implant. In some embodiments, prior to complete curing of the molten material, an infection control composition, for example a powder, may be adhered to a surface of the implant (instead of or in addition to mixing the porphyrin with the biocompatible material). Alternatively, the method may include monitoring a temperature of the mixture until it reaches a predefined temperature range (e.g., between about 40 degrees Celcius and about 70 degrees Celcius, less than about 70 degrees Celcius, less than about 40 degrees Celcius, between about 200 degrees Celcius and about 400 degrees Celcius, less than about 200 degrees Celcius, less than about 400 degrees Celcius, etc.) and adding in an infection control composition after the mixture is in the predefined temperature range.

The method 1500 may optionally further include creating a wax pattern, for example, using a mold created by additive manufacturing.

The method 1500 may optionally further include assembling two or more patterns into a wax sprue to allow multiple parts to be cast simultaneously.

The method 1500 may optionally further include dipping the assembly into a ceramic slurry and coating the assembly with sand.

The method 1500 may optionally further include heating the assembly to cause the wax to melt, leaving behind a hollow ceramic shell.

The method 1500 may optionally further include pouring the biocompatible material into the hollow ceramic mold (i.e., filling the spaces where the wax once was).

The method 1500 may optionally further include cooling the biocompatible material and/or allowing the biocompatible material to solidify within the ceramic mold.

The method 1500 may optionally further include removing the ceramic shell to reveal the cast implant.

The method 1500 may optionally further include post-processing the implant, for example, grinding, polishing, and/or machining to achieve the desired specifications and surface finish.

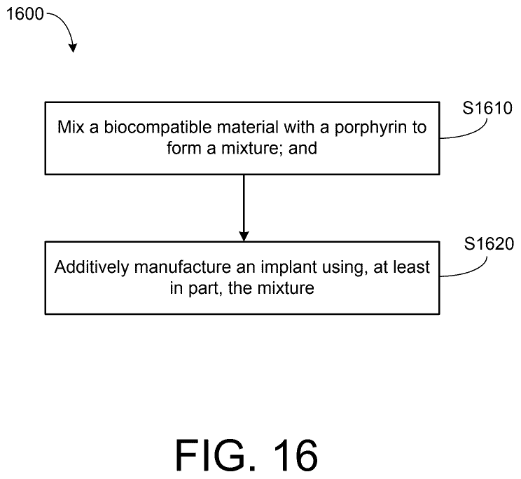

FIG. 16 shows an embodiment of a method 1600 for additively manufacturing an infection resistant implant. The method includes mixing a biocompatible material with a porphyrin to form a mixture at block S1610; and additively manufacturing an implant using, at least in part, the mixture at block S1620. Method 1600 may be used to create orthopedic implants (e.g., hip, knee, shoulder), spinal implants, bone plates, bone screws, dental implants (e.g., dental crowns, bridges, other prosthetic components), cardiovascular devices (e.g., stents, heart valve components, other cardiovascular implants), and/or custom implants. The implants may be suitable for patient-specific implants that require bespoke design and precision.

As shown in FIG. 16, an embodiment of a method 1600 for additively manufacturing an infection resistant implant includes block S1610, which recites mixing a biocompatible material with a porphyrin to form a mixture. The biocompatible material may include titanium, a stainless steel, a titanium alloy, a ceramic, a cobalt chromium alloy, an oxidized zirconium alloy, bone cement, polyethylene, a biocompatible polymer, and the like. Mixing may include soaking the material in the infection control composition, adding the infection control composition to the material when it is in a liquid state, or otherwise bonding the infection control mixture to or into the biocompatible material.

As shown in FIG. 16, an embodiment of a method 1600 for additively manufacturing an infection resistant implant includes block S1620, which recites additively manufacturing an implant using, at least in part, the mixture. Additive manufacturing may include selective laser melting (SLM) which uses a high-powered laser to melt and fuse metal powder. Additive manufacturing may include electron beam melting (EBM) which uses an electron beam to melt and fuse metal powder. Additive manufacturing may include stereolithography (SLA) which uses a laser to cure and solidify photopolymer resin. Additive manufacturing may include fused deposition modeling (FDM) which extrudes thermoplastic filament layer by layer.

The method 1600 may optionally further include receiving a three-dimensional image or scan of an anatomical region of a patient. The image or scan may be received from a magnetic resonance imager (MRI), a computer tomography scan, or a 3D scan. The method 1600 may optionally further include generating a digital model of the implant based on the anatomical data, image, or scan. The method 1600 may optionally further include converting the digital model into a series of layers using slicing software. Each layer represents a cross-section of the implant.

The method 1600 may optionally further include post-processing the implant. Post-processing may include removing supports used during printing, applying a heat treatment (e.g., annealing) to improve the material properties, and/or applying a surface finish (e.g., polish, machine, or coat) to the implant to achieve the desired surface finish and/or increase biocompatibility.

### EXAMPLES

Example 1. A method of reducing biofilm formation on an implant, comprising: soaking an orthopedic implant comprising a biocompatible material in an infection control composition comprising a porphyrin.

Example 2. The method of any one of the preceding examples, but particularly Example 1, wherein the orthopedic implant is formed by one of: sintering, casting, injection molding, or machining.

Example 3. The method of any one of the preceding examples, but particularly Example 1, wherein the biocompatible material comprises one or more of: a stainless steel, a titanium alloy, a ceramic, a cobalt chromium alloy, an oxidized zirconium alloy, bone cement, polyethylene, or a polymer.

Example 4. The method of any one of the preceding examples, but particularly Example 1, wherein the orthopedic implant comprises one of: a joint implant, a bone plate, an intramedullary rod, a bone graft, a spinal implant, or a dental implant.

Example 5. The method of any one of the preceding examples, but particularly Example 1, wherein the porphyrin comprises:

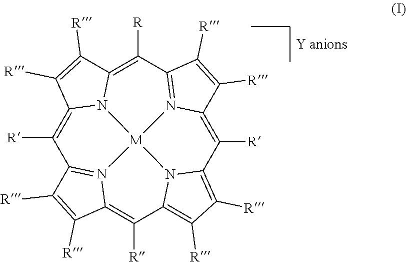

wherein, M is a transition metal (II) cation, R is a mono-, di-, tri-, tetra-, or penta-halophenyl and the halogen is selected from the group consisting of Cl, F, Br, and combinations thereof, R′ is

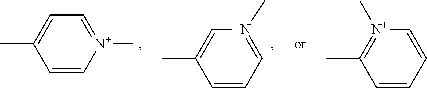

R″ is equal to R or R′, R″ is —H, —F, —Cl, or —Br, and Y is 2 or 3.

Clause 6. The method of any one of the preceding examples, but particularly Example 5, wherein the transition metal M is a Fe⁺², a Co⁺², a Ni⁺², or Zn⁺².

Example 7. The method of any one of the preceding examples, but particularly Example 6, wherein the porphyrin comprises:

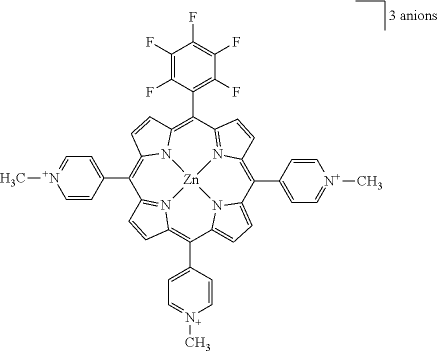

and wherein the anion is p-toluene sulfonate.

Example 8. The method of any one of the preceding examples, but particularly Example 1, wherein the porphyrin comprises a transition metal porphyrin complex having, in an absence of light, one or more of: antimicrobial activity, antibacterial activity, or antiviral activity.

Example 9. The method of any one of the preceding examples, but particularly Example 1, wherein the infection control composition further comprises a pharmaceutically acceptable excipient.

Example 10. The method of any one of the preceding examples, but particularly Example 9, wherein a weight ratio of the porphyrin to the pharmaceutically acceptable excipient is about 1:1,000 to about 1:500,000.

Example 11. The method of any one of the preceding examples, but particularly Example 9, wherein a weight ratio of the porphyrin to the pharmaceutically acceptable excipient is about 1:2,000 to about 1:250,000.

Example 12. The method of any one of the preceding examples, but particularly Example 9, wherein the pharmaceutically acceptable excipient comprises one or more of: saline, water, a disinfectant, or a combination thereof.

Example 13. The method of any one of the preceding examples, but particularly Example 1, wherein an effective concentration of the porphyrin in the infection control composition is about 2 μg/mL to about 1000 μg/mL.

Example 14. The method of any one of the preceding examples, but particularly Example 1, wherein an effective concentration of the porphyrin in the infection control composition is about 4 μg/mL to about 500 μg/mL.

Example 15. The method of any one of the preceding examples, but particularly Example 1, wherein the orthopedic implant is light activated before implantation.

Example 16. The method of any one of the preceding examples, but particularly Example 15, wherein the light has a wavelength of about 400 nm to about 850 nm.

Example 17. The method of any one of the preceding examples, but particularly Example 1, further comprising reducing biofilm formation on the implant.

Example 18. An implant for implantation in an animal body, comprising: an implant body comprising a biocompatible material and defining one or more reservoirs; and an infection control composition comprising a porphyrin composition configured to be retained in the one or more reservoirs, wherein the one or more reservoirs are configured to elute the porphyrin composition over time.

Example 19. The implant of any one of the preceding examples, but particularly Example 18, wherein the biocompatible material comprises one or more of: a stainless steel, a titanium alloy, a ceramic, a cobalt chromium alloy, an oxidized zirconium alloy, bone cement, polyethylene, or a polymer.

Example 20. The implant of any one of the preceding examples, but particularly Example 18, wherein the implant comprises one of: a joint implant, a bone plate, an intramedullary rod, an intramedullary nail, a bone graft, a spinal implant, or a dental implant.

Example 21. The implant of any one of the preceding examples, but particularly Example 18, wherein the porphyrin comprises:

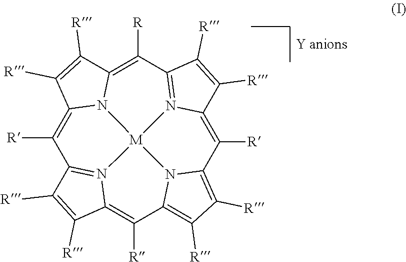

wherein, Mis a transition metal (II) cation, R is a mono-, di-, tri-, tetra-, or penta-halophenyl and the halogen is selected from the group consisting of Cl, F, Br, and combinations thereof, R′ is

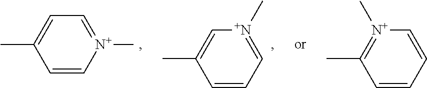

R″ is equal to R or R′, R″ is —H, —F, —Cl, or —Br, and Y is 2 or 3.

Example 22. The implant of any one of the preceding examples, but particularly Example 21, wherein the transition metal M is a Fe⁺², a Co⁺², a Ni⁺², or Zn⁺².

Example 23. The implant of any one of the preceding examples, but particularly Example 22, wherein the porphyrin composition comprises:

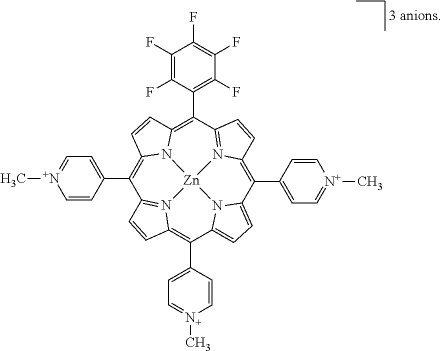

wherein the anion is p-toluene sulfonate.

Example 24. The implant of any one of the preceding examples, but particularly Example 18, wherein the porphyrin comprises a transition metal porphyrin complex having, in an absence of light, one or more of: antimicrobial activity, antibacterial activity, or antiviral activity.

Example 25. The implant of any one of the preceding examples, but particularly Example 18, wherein a volume of a reservoir of the one or more reservoirs defined by the implant body is about 1 mL to about 10 mL.

Example 26. The implant of any one of the preceding examples, but particularly Example 18, wherein a concentration of the porphyrin composition in the infection control composition is about 2 μg/mL to about 1000 μg/mL.

Example 27. The implant of any one of the preceding examples, but particularly Example 18, wherein a concentration of the porphyrin composition in the infection control composition is about 4 μg/mL to about 500 g/mL.

Example 28. The implant of any one of the preceding examples, but particularly Example 18, wherein an external surface of the implant body is substantially resistant to biofilm formation for about one week to about six months.

Example 29. The implant of any one of the preceding examples, but particularly Example 18, wherein the implant body is porous.

Example 30. The implant of any one of the preceding examples, but particularly Example 29, wherein a porosity of the implant body is about 30% to about 80%.

Example 31. An implant for implantation in an animal body, comprising: an implant body comprising a biocompatible material, the implant body having a hydroxyapatite coating on at least a portion of an exterior surface of the implant body; and an infection control composition comprising porphyrin configured to react with or bond to the hydroxyapatite coating, wherein the porphyrin is configured to reduce biofilm formation on the implant body.

Example 32. A method of reducing biofilm formation on an implant, comprising: reaming an intramedullary canal of a bone; applying an infection control composition comprising porphyrin to the reamed intramedullary canal; implanting an intramedullary nail into the intramedullary canal; and reducing infection or biofilm formation on the intramedullary nail.

Example 33. A method of manufacturing an implant, comprising: mixing a biocompatible material with an infection control composition comprising porphyrin to form a mixture; and casting the mixture in a mold to form an orthopedic implant.

Example 34. A method of manufacturing an implant comprising: mixing a biocompatible material with an infection control composition comprising porphyrin to form a mixture; and additively manufacturing an implant using, at least in part, the mixture.

Example 35. The method of any one of the preceding examples, but particularly Example 34, wherein the additively manufacturing is selective laser melting, electron beam melting, stereolithography, or fused deposition modeling.

Example 36. A method of disinfecting an implant, comprising: applying an infection control composition to an external surface of an orthopedic implant, the infection control composition comprising a porphyrin and a pharmaceutically acceptable excipient.

Example 37. The method of any one of the preceding examples, but particularly Example 32, further comprising activating the porphyrin with light having a wavelength between about 400 nm to about 850 nm.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “excipient” may include, and is contemplated to include, a plurality of excipients. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implant for implantation in an animal body, comprising:
an implant body comprising a biocompatible material and defining one or more reservoirs; and
an infection control composition comprising a porphyrin composition configured to be retained in the one or more reservoirs,
wherein the one or more reservoirs are configured to elute the porphyrin composition over time.

2. The implant of claim 1, wherein the biocompatible material comprises one or more of: a stainless steel, a titanium alloy, a ceramic, a cobalt chromium alloy, an oxidized zirconium alloy, bone cement, polyethylene, or a polymer.

3. The implant of claim 1, wherein the implant comprises one of: a bone screw, a joint implant, a bone plate, an intramedullary rod, an intramedullary nail, a bone graft, a spinal implant, or a dental implant.

4. The implant of claim 1, wherein the porphyrin composition comprises a porphyrin comprising: 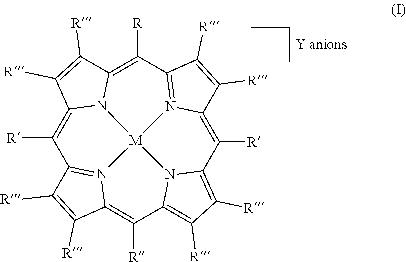
wherein,
M is a transition metal (II) cation,
R is a mono-, di-, tri-, tetra-, or penta-halophenyl and the halogen is selected from the group consisting of Cl, F, Br, and combinations thereof,
R′ is 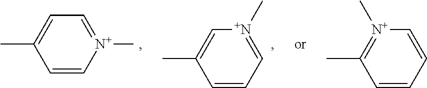
R″ is equal to R or R′,
R″ is —H, —F, —Cl, or —Br, and
Y is 2 or 3.

5. The implant of claim 4, wherein the transition metal M is a Fe⁺², a Co⁺², a Ni⁺², or Zn⁺².

6. The implant of claim 5, wherein the porphyrin composition comprises: 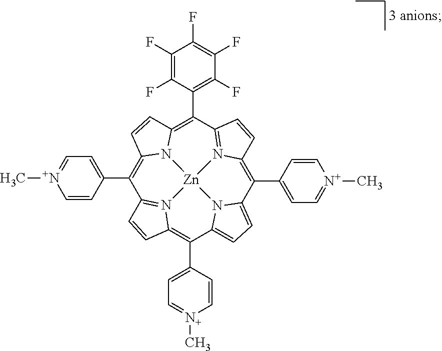
wherein the anion is p-toluene sulfonate.

7. The implant of claim 1, wherein the porphyrin composition comprises a transition metal porphyrin complex having, in an absence of light, one or more of: antimicrobial activity, antibacterial activity, or antiviral activity.

8. The implant of claim 1, wherein a volume of a reservoir of the one or more reservoirs defined by the implant body is about 1 ml to about 10 ml.

9. The implant of claim 1, wherein a concentration of the porphyrin composition in the infection control composition is about 2 μg/ml to about 1000 μg/ml.

10. The implant of claim 1, wherein a concentration of a porphyrin in the porphyrin composition in the infection control composition is about 4 μg/ml to about 500 μg/ml.

11. The implant of claim 1, wherein an external surface of the implant body is substantially resistant to biofilm formation for about one week to about six months.

12. The implant of claim 1, wherein the implant body is porous.

13. The implant of claim 12, wherein a porosity of the implant body is about 30% to about 80%.

14. The implant of claim 1, wherein the implant is formed by one of: sintering, casting, injection molding, or machining.

15. The implant of claim 1, wherein the infection control composition further comprises a pharmaceutically acceptable excipient.

16. The implant of claim 15, wherein a weight ratio of the porphyrin composition to the pharmaceutically acceptable excipient is about 1:1,000 to about 1:500,000.

17. The implant of claim 15, wherein a weight ratio of the porphyrin composition to the pharmaceutically acceptable excipient is about 1:2,000 to about 1:250,000.

18. The implant of claim 15, wherein the pharmaceutically acceptable excipient comprises one or more of: saline, water, a disinfectant, or a combination thereof.

19. The implant of claim 1, wherein the infection control composition is configured to be light activated while retained in the reservoir.

20. The implant of claim 1, wherein the infection control composition is configured to be light activated prior to deposition in the reservoir.
